(19) United States
(12) Patent Application Publication
(10) Pub. No.: US 2010/0241158 A1
(43) Pub. Date: Sep. 23, 2010

(54) ACUPUNTURE DEVICE

(76) Inventor: Aharon HERMON, Kfar Monash (IL)

Correspondence Address: The Law Office of Michael E. Kondoudis, 888 16th Street, N.W., Suite 800, Washington, DC, 20006, US

(21) Appl. No.: 12/728,286

(22) Filed: Mar. 22, 2010

Related U.S. Application Data
(60) Provisional application No. 61/162,298, filed on Mar. 22, 2009.

(51) Int. Cl. A61B 17/00

(52) U.S. Cl. 606/204

(57) ABSTRACT

An acupuncture device for treating a patient comprising an acupuncture needle connected to a holder. The holder is arranged to allow placing and fixating the acupuncture needle at a predefined acupoint on the user's outer ear, for example on an acupoint related to appetite suppression. The device may be used for a long term without displacement of the needle. The device may be placed with a spring loaded forceps, arranged to open and close the span of the holder.

### CROSS REFERENCE TO RELATED APPLICATIONS

This application claims the benefit of U.S. Provisional Patent Application 61/162,298 filed on Mar. 22, 2009, which is incorporated herein by reference.

### BACKGROUND

1. Technical Field

The present invention relates to the field of treatment supporting appliances, and more particularly, to appliances utilizing acupuncture.

2. Discussion of Related Art

An effective diet is a major concern to many people. There are very many methods for reducing weight, some of them utilize acupuncture treatment at a specialized clinic. Moreover, various ailments may be handled by acupuncture. However, placing acupuncture needles requires both knowledge and skill

### BRIEF SUMMARY

Embodiments of the present invention provide an acupuncture device comprising: a U-shaped holder comprising: a placing arm having a connector end; and a fixating arm having a supportive end, the fixating arm being longer than the placing arm, wherein the placing arm and the fixating arm are connected by a curvature having a user adjustable angle. The acupuncture device further comprises a treatment element connected to the holder at the connector end, and arranged to treat a user when placed at a specified acupoint. The holder is arranged to allow placing the treatment element at the specified acupoint on the user's outer ear by spanning the holder at a first angle range of the user adjustable angle, and further arranged to allow fixating the treatment element at the specified acupoint on the user's outer ear, at a second angle range of the user adjustable angle, against the supportive end.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

The present invention will be more readily understood from the detailed description of embodiments thereof made in conjunction with the accompanying drawings of which:

FIG. 1 is a high level scheme of an ear with an attached device for enhancing a diet, according to some embodiments of the invention;

FIGS. 2A, 2B, and 2C are schematic illustrations of the acupuncture device, according to some embodiments of the invention;

FIGS. 3A and 3B are schematic illustrations of user adjustment of the user adjustable angle, according to some embodiments of the invention; and

FIGS. 4A and 4B are high level flowcharts of methods, according to some embodiments of the invention.

### DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

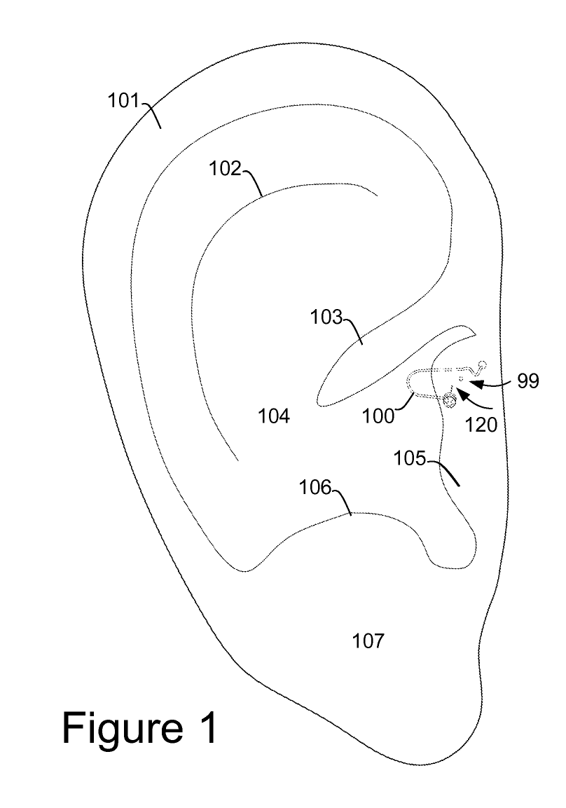

FIG. 1 is a schematic illustration of an ear with an attached acupuncture device 100 for enhancing a diet, according to some embodiments of the invention. The parts of the ear are marked as following: a helix 101, an anti-helix 102, a crus 103, a concha 104, a tragus 105, an anti-tragus 106, a lobula 107. Acupuncture device 100 comprises an acupuncture needle 120 connected to a U-shaped holder 110.

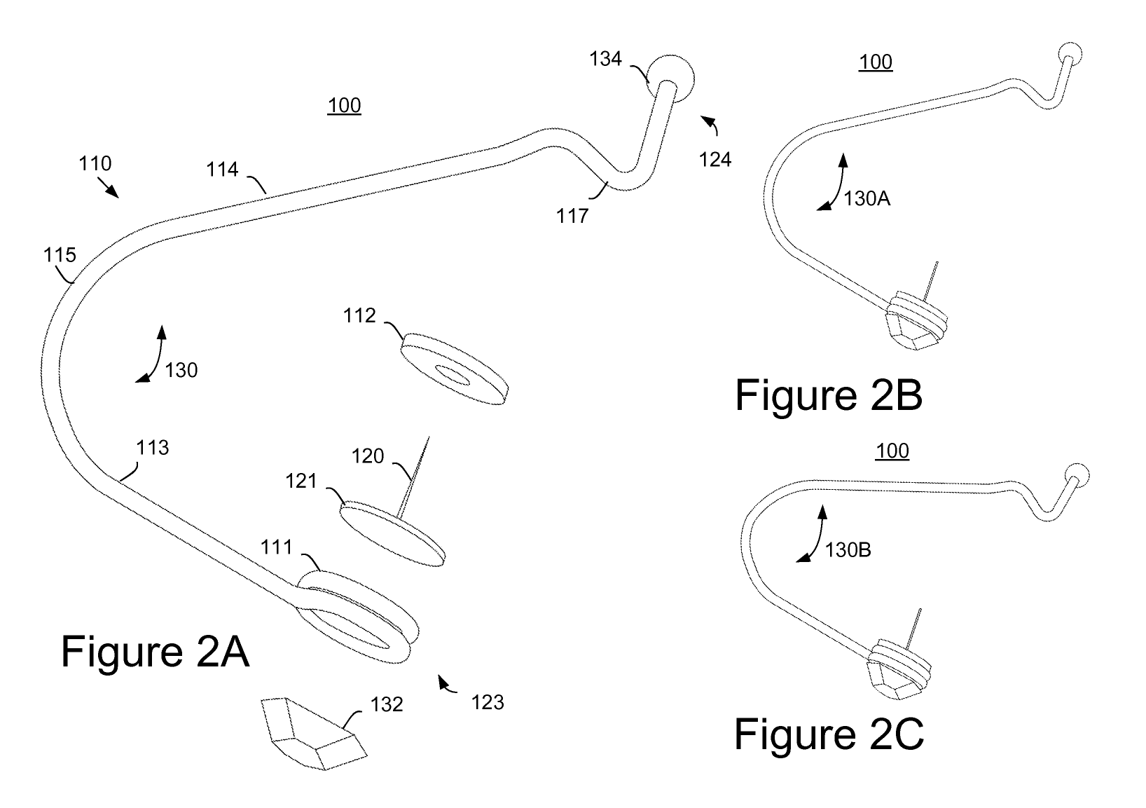

FIGS. 2A, 2B, and 2C are schematic illustrations of acupuncture device 100, according to some embodiments of the invention. FIG. 2A is an exploded view of device 100, FIGS. 2B and 2C illustrate two states of device 100, according to some embodiments of the invention.

U-shaped holder 110 comprises a placing arm 113 having a connector end 123, and a fixating arm 114 having a supportive end 124. Fixating arm 114 is longer than placing arm 113, the lengths of placing arm 113 and of fixating arm 114 may be configured according to an ear structure of the user.

Placing arm 113 and fixating arm 114 are connected by a curvature 115 having a user adjustable angle 130. Curvature 115 may be flexible and allow some elastic and some plastic deformation such as to allow a user adjust angle 130.

Acupuncture device 100 further comprises a treatment element such as acupuncture needle 120 connected to holder 110 at connector end 123. The treatment element is arranged to treat a user when placed at a specified acupoint.

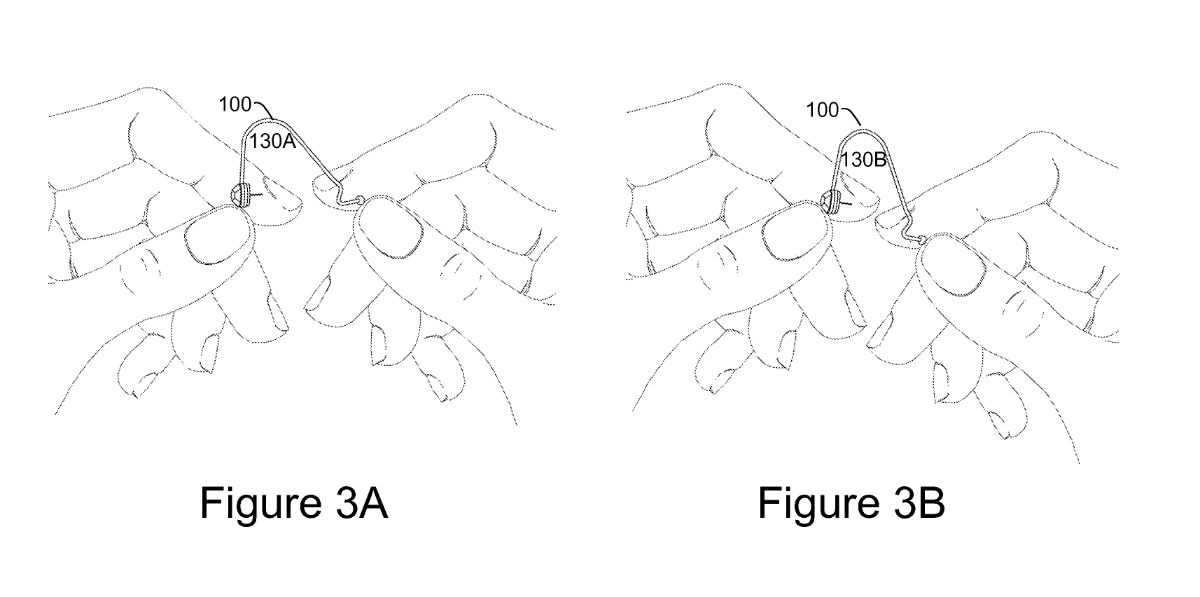

FIGS. 3A and 3B are schematic illustrations of user adjustment of angle 130, according to some embodiments of the invention. Angle 130 is adjusted such as to fixate device 100 on the user's ear with needle 120 in its correct place.

Holder 110 is arranged to allow placing the treatment element at the specified acupoint on the user's outer ear by spanning holder 110 at a first angle range 130A, for example, circa 45°, of user adjustable angle 130, and further arranged to allow fixating the treatment element at the specified acupoint on the user's outer ear, at a second angle range 130B, for example, circa 30°, of user adjustable angle 130, against supportive end 124.

Supportive end 124 may comprises a fold 117 arranged to stabilize the treatment element in place. Supportive end 124 may further comprise a bead 134 connected to supportive end 124. Bead 134 may have a decorative value and further conceal the treatment element.

In case the treatment element is acupuncture needle 120, U-shaped holder 110 is arranged to allow placing and fixating acupuncture needle 120 at a predefined acupoint on tragus 106 of the user's outer ear. Acupuncture needle 120 may comprise a basis 121 allowing an effective and long lasting attachment to holder 110. Acupuncture needle 120 may be attached to holder 110 using any method, for example, using laser welding or laser soldering techniques. Acupuncture needle 120 may be attached to holder 110 by a glue compatible for long contact with the skin. Acupuncture needle 120 may be attached to holder 110 by producing device 100 as one piece (i.e., acupuncture needle 120 may be formed during production of holder 110).

According to some embodiments of the invention, device 100 may be applied to other acupoints on the ear for treating various conditions or diseases, such as acupoints relating to skeletal elements or to inner organs.

According to some embodiments of the invention, device 100 may comprise elements for applying an acupuncture treatment other that acupuncture needle 120. As an example—a magnet (not shown) may be attached to holder 110, such that the magnet contacts a selected acupoint on the ear. In another embodiment, a pressure applying element (not shown) may be connected to holder 110, such as to apply a predefined pressure onto a selected acupoint. In yet another embodiment, a electric element (not shown) may be connected to holder 110, and arranged to stimulate the selected acupoint. Any other treating element may be connected to holder 110.

According to some embodiments of the invention, holder 110 may be arranged to allow placing and fixating acupuncture needle 120 at a predefined acupoint 99 on tragus 106 of the user's outer ear. The form and elasticity of holder 110 may be configured such as to fit and hold on the location on the ear that is selected for applying device 100. Predefined acupoint 99 may be selected such as to enhance a diet, for example by suppressing appetite. Predefined acupoint 99 may be selected on tragus 106, for example as T5, or any other acupoint of the ear relating to any part of the body. In case of T5, the form and elasticity of holder 110 may be configured such as to fit and hold on tragus 106 and facilitate easy placing of device 100. In other cases, the structure of holder 110 may be shaped to facilitate correct placing of device 100, for example the size of holder 110 may be made to fit thereto. In another example, the lengths of arms 113, 114 of holder 110 may be adjusted according to the selected acupoint and the form of the treated ear. Device 100 may be placed with a spring loaded forceps, arranged to open and close span 130 of holder 110. A special spring loaded forceps may be designed or configured to fit to either device 100, the user's ear structure or both. Device 100 may be placed by hand, and a correct placement may be facilitated by the size and form of holder 110.

FIG. 2 is a high level scheme of device 100 for enhancing a diet, according to some embodiments of the invention. Device 100 comprises acupuncture needle 120 connected to holder 110 exhibiting span 130. Acupuncture needle 120 may comprise a standard press needle. Acupuncture needle 120 may comprise basis 121 allowing an effective and long lasting attachment to holder 110. Holder 110 may further comprise a spring (not shown) configured to facilitate placing device 100 (for example with a spring loaded forceps). Holder 110 may further comprise supportive fold 117 arranged to contact the back side of tragus 106, opposing acupuncture needle 120. Device 100 may be arranged to allow long term use of device 100, for example for weeks and months of treatment, without the risk nor need to displace acupuncture needle 120 from predefined location 99. Device 100 may be arranged to allow short term use of device 100. Device 100 may be arranged to be placed at other locations on the ear and at other locations of the body for treating different maladies and complaints of the user.

According to some embodiments of the invention, connector end 123 of holder 110 may comprise a basal loop 111, to which basis 121 of acupuncture needle 120 is attached. A perforated disc 112 may be attached upon acupuncture needle 120, such that acupuncture needle 120 fits through the perforation in perforated disc 112. Basal loop 111, basis 121 of acupuncture needle 120, and perforated disc 112 may be fastened together, e.g., by laser welding, or gluing.

According to some embodiments of the invention, the dimensions of device 100 may be adapted to facilitate easy placing of acupuncture needle 120 in optimal safety and accuracy. Various parameter such as the length of holder 110, lengths of placing arm 113 and of fixating arm 114, user adjustable angle 130 and first and second angle ranges, and flexibility of holder 110 and curvature 115 may be adjusted according to treatment and patient requirements.

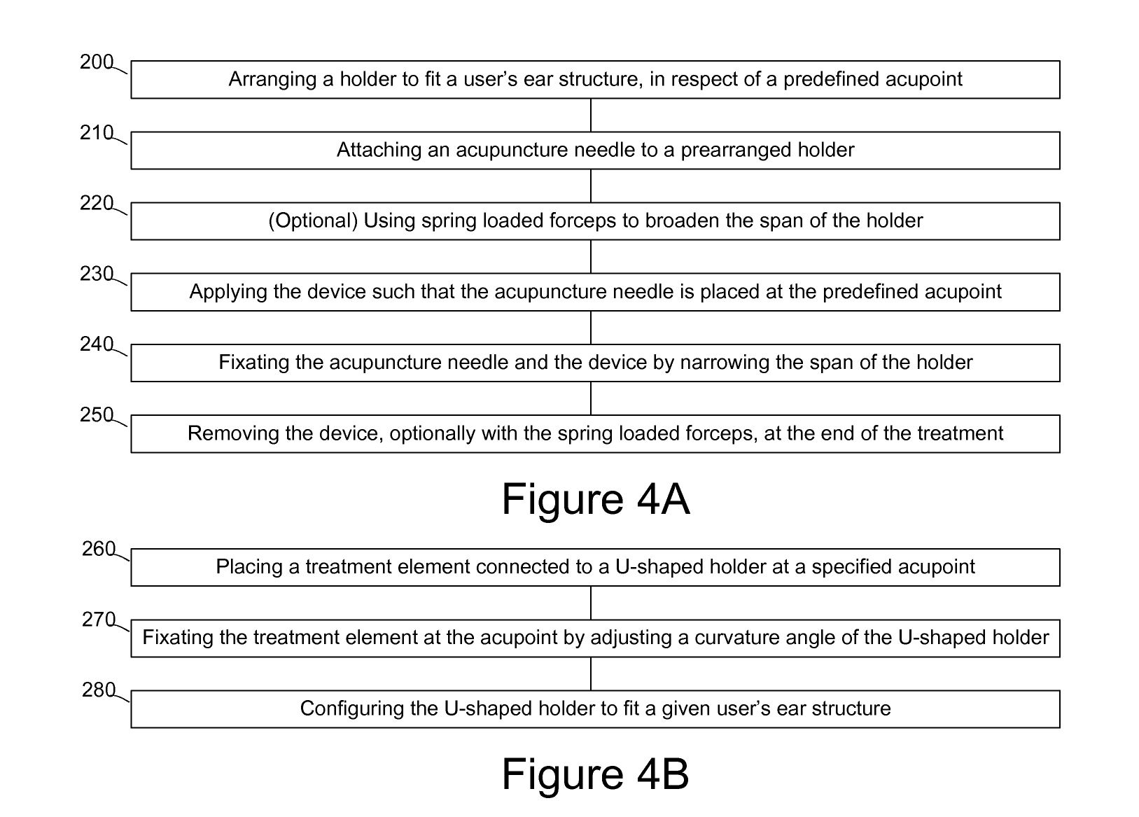

FIG. 4A is a high level flowchart of a method of enhancing a diet, according to some embodiments of the invention. The method may comprise some or all of the following stages, some or all of which may be carried out by either users, suppliers or treatment personnel: arranging a holder to fit a user's ear structure, in respect of a predefined acupoint (stage 200); attaching an acupuncture needle to a prearranged holder (stage 210); optionally using spring loaded forceps to broaden the span of the holder (stage 220); applying the device such that the acupuncture needle is placed at the predefined acupoint (stage 230); fixating the acupuncture needle and the device by narrowing the span of the holder (stage 240); and removing the device, optionally with the spring loaded forceps, at the end of the treatment (stage 250). The device may be placed and removed by hand, or utilizing an appliance. The device may be placed by a physician or by the user, and may be constructed such as to allow comfortable and exact placement.

According to some embodiments of the invention, the method may be accompanied by a dietary plan complementing the acupuncture treatment.

According to some embodiments of the invention, both device 100 and the method may be applied for treatments other than enhancing diet. Such treatments may comprise relaxing, treating various physiological or psychological disorders, relieving pains, and the like.

Embodiments of the present invention allow safe and effective long term application of acupuncture needles and allow users to effectively place on their own acupuncture needles at predefined locations.

FIG. 4B is a high level flowchart of a method, according to some embodiments of the invention. The method comprises: placing a treatment element connected to a first end of a U-shaped holder at a specified acupoint (stage 260); and fixating the treatment element at the specified acupoint by adjusting a curvature angle of the U-shaped holder, such that a second end of the U-shaped holder stabilizes the treatment element at the specified acupoint (stage 270). Adjusting the curvature angle may be carried out using a spring loaded forceps or manually. The specified acupoint may be selected such as to enhance a diet, or to treat other patient's complaints. The method may further comprise configuring the U-shaped holder to fit a given user's ear structure (stage 280).

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claimed is:

1. An acupuncture device comprising:
a U-shaped holder comprising:
a placing arm having a connector end; and
a fixating arm having a supportive end, the fixating arm being longer than the placing arm,
wherein the placing arm and the fixating arm are connected by a curvature having a user adjustable angle, and
a treatment element connected to the holder at the connector end, and arranged to treat a user when placed at a specified acupoint,
wherein the holder is arranged to allow placing the treatment element at the specified acupoint on the user's outer ear by spanning the holder at a first angle range of the user adjustable angle, and further arranged to allow fixating the treatment element at the specified acupoint on the user's outer ear, at a second angle range of the user adjustable angle, against the supportive end.

2. The acupuncture device of claim 1, wherein the treatment element comprises an acupuncture needle.

3. The acupuncture device of claim 2, wherein the acupuncture needle is connected to the connector end by at least one of: gluing; laser welding; laser soldering; and forming during production of the holder.

4. The acupuncture device of claim 2, wherein the connector end comprises a basal loop, wherein the acupuncture needle comprises a basis, and wherein the basis is connected to the basal loop by fastening a perforated disc thereupon, such that the acupuncture needle fits through the perforation in the perforated disc.

5. The acupuncture device of claim 1, wherein the specified acupoint comprises T5.

6. The acupuncture device of claim 1, wherein the treatment element comprises at least one of: a magnet; an electric element; and a pressure applying element.

7. The acupuncture device of claim 1, further comprising a decorative element attached to the connector end opposite to the treatment element, such that the decorative element is visible to a person facing the user.

8. The acupuncture device of claim 7, wherein the decorative element is a gemstone.

9. The acupuncture device of claim 1, wherein the supportive end comprises a fold arranged to stabilize the treatment element in place.

10. The acupuncture device of claim 1, further comprising a bead connected to the supportive end.

11. The acupuncture device of claim 1, wherein the first angle range of the user adjustable angle is substantially 45° and wherein the second angle range is substantially 30°.

12. The acupuncture device of claim 1, wherein lengths of the placing arm and the fixating arm are configured according to an ear structure of the user.

13. A method, comprising:
placing a treatment element connected to a first end of a U-shaped holder at a specified acupoint; and
fixating the treatment element at the specified acupoint by adjusting a curvature angle of the U-shaped holder, such that a second end of the U-shaped holder stabilizes the treatment element at the specified acupoint.

14. The method of claim 13, wherein the specified acupoint is selected such as to enhance a diet.

15. The method of claim 13, wherein adjusting the curvature angle is carried out using a spring loaded forceps.

16. The method of claim 13, wherein adjusting the curvature angle is carried out manually.

17. The method of claim 13, further comprising configuring the U-shaped holder to fit a given user's ear structure.

18. An acupuncture device comprising:
a U-shaped holder comprising:
a placing arm having a connector end comprising a basal loop; and
a fixating arm having a supportive end comprising a fold and a bead, the fixating arm being longer than the placing arm, and lengths of the placing arm and the fixating arm are configured according to an ear structure of a user,
wherein the placing arm and the fixating arm are connected by a curvature having a manually adjustable angle,
an acupuncture needle comprising a basis that is connected to the basal loop by fastening a perforated disc thereupon, such that the acupuncture needle fits through the perforation in the perforated disc.
a gemstone attached to the connector end opposite to the acupuncture needle, such that the gemstone is visible from to a person facing the user;
wherein the holder is arranged to allow placing the acupuncture needle at T5 acupoint on the user's outer ear by spanning the holder at a first angle range of the adjustable angle, and further arranged to allow fixating the acupuncture needle at T5, at a second angle range of the adjustable angle, against the supportive end, such that the fold stabilizes the acupuncture needle at T5.
